(19) United States
(12) Patent Application Publication
(10) Pub. No.: US 2007/0282191 A1
(43) Pub. Date: Dec. 6, 2007

(54) ENDOSCOPIC ULTRASOUND FOR THORACIC DUCT LYMPH COLLECTION

(76) Inventor: Vinod K. Parasher, Rehobeth Beach, DE (US)

Correspondence Address: Connolly Bove Lodge & Hutz LLP, P.O. Box 2207, Wilmington, DE, 19899-2207, US

(21) Appl. No.: 11/751,881

(22) Filed: May 22, 2007

Related U.S. Application Data
(60) Provisional application No. 60/809,521, filed on May 31, 2006.

(51) Int. Cl. A61B 5/05; A61B 1/00

(52) U.S. Cl. 600/409; 600/102

(57) ABSTRACT

A method of puncturing the thoracic duct by endoscopic ultrasound is performed on humans to collect specimens from the thoracic duct lymph.

### CROSS-REFERENCE TO RELATED APPLICATION

This application is based on provisional application Ser. No. 60/809,521 filed May 31, 2006, all of the details of which are incorporated herein by reference thereto.

### BACKGROUND OF INVENTION

I have described in the article “Lymph sampling and lymphangiography via EUS-guided transesophageal thoracic duct puncture in a swine model”; Gastrointestinal Endoscopy Vol. 59, No. 4, 2004 (pp 564-67) techniques that have been used for the lymph sampling and lymphangiography via endoscopic guided transesophageal thoracic duct puncture studies in porcine models where the thoracic duct under endoscopic ultrasound guidance allows lymph sampling and analysis. As pointed out in that article, although the mechanism of formation and transport of lymph have been studied in animals, knowledge of the generation and regulation of lymph flow in humans is limited.

### SUMMARY OF INVENTION

An object of this invention is to apply the techniques of the above noted article for use in humans.

### DETAILED DESCRIPTION

The present invention incorporates techniques described in that article. All of the details of that article (a copy of which is attached) are incorporated herein by reference thereto. See in particular the DISCUSSION section of that article. The above noted article shows the feasability of being able to use EUS-guided techniques for lymph sampling in swine. The present invention goes beyond the discussions in that article by not only applying such techniques for human purposes, but also for utilizing the techniques in humans to obtain specimens or samples which could then be diagnosed as later described. In particular this invention for the first time utilizes endoscopic ultrasound to collect lymph in humans and could be practiced for all applications resulting from such collection of lymph.

Such techniques using endoscopic ultrasound guidance can be done on humans to obtain specimens which can be subjected to molecular biology for investigating, for example, cancer cells or bacterial tumor biology. This marks a distinct advancement in the art since there is no simple access to the thoracic duct lymph collection in humans. The endoscopic ultrasound guided puncture may not only provide a much needed access, but also the lymph could be sent for analysis to pick up cancer cells and evaluate early diagnosis or therapy and study immunology. For example, the lymph itself could be analyzed for a normal patient and for a patient with cancer. In analyzing the lymph the study for cancer could use markers that are now, for example, used for blood study.

What is claimed is:

1. A method of puncturing the thoracic duct by endoscopic ultrasound wherein the method is performed on humans to collect specimens from the thoracic duct lymph.

2. The method of claim 1 wherein the lymph itself is provided for analysis to pick up cancer cells.

3. The method of claim 2 wherein the lymph is evaluated for early diagnosis of cancer.

4. The method of claim 2 wherein the lymph is evaluated for therapy of cancer.

5. The method of claim 2 wherein the lymph is evaluated for study immunology.

6. The method of claim 1 wherein the specimens are analyzed for diagnosis thereof.
